(19) United States
(12) Patent Application Publication
(10) Pub. No.: US 2023/0330233 A1
(43) Pub. Date: Oct. 19, 2023

(54) COMPOSITIONS AND METHODS FOR DELIVERING PHARMACEUTICAL AGENTS

(71) Applicant: The Regents of the University of Michigan, Ann Arbor, MI (US)

(72) Inventors: Adam J. Matzger, Ann Arbor, MI (US); Suresh Kuthuru, Ann Arbor, MI (US)

(21) Appl. No.: 18/116,637

(22) Filed: Mar. 2, 2023

Related U.S. Application Data
(62) Division of application No. 16/688,221, filed on Nov. 19, 2019, now abandoned.
(60) Provisional application No. 62/769,760, filed on Nov. 20, 2018.

(51) Int. Cl. A61K 47/24; A61K 31/519; A61K 31/192; A61K 31/12

(52) U.S. Cl. CPC A61K 47/24; A61K 31/519; A61K 31/192; A61K 31/12

(57) ABSTRACT

Provided herein are compositions and method for improving drug solubility and delivery. In particular, provided herein are metal-organic frameworks and their use in improving drug solubility and delivery.

This application is a divisional of U.S. patent application Ser. No. 16/688,221, filed Nov. 19, 2019, which claims priority to U.S. provisional patent application Ser. No. 62/769,760, filed Nov. 20, 2018 which is incorporated herein by reference in its entirety.

### STATEMENT REGARDING FEDERALLY SPONSORED RESEARCH OR DEVELOPMENT

This invention was made with government support under DE-SC0004888 awarded by the Department of Energy. The government has certain rights in the invention.

### FIELD OF THE DISCLOSURE

Provided herein are compositions and method for improving drug solubility and delivery. In particular, provided herein are metal-organic frameworks and their use in improving drug solubility and delivery.

### BACKGROUND

Oral delivery is the preferred route for drug administration due to convenience, high patient compliance, and cost-effectiveness. This route of administration benefits from fast drug dissolution in the gastrointestinal tract, which results in relatively rapid absorption and the accompanying onset of therapeutic activity (Sun, D. D.; Lee, P. I. Mol. Pharmaceutics 2013, 10, 4330-4346). Achieving the desired concentration to facilitate drug absorption depends on solubility in physiological conditions, and this can be a significant challenge in drug development and formulation of lipophilic drugs. Drug candidates being generated in the R&D pipeline and several marketplace drugs often suffer from poor solubility (Fahr, A.; Liu, X. Expert. Opin. Drug Del. 2007, 4, 403-416; Paul, S. M. et al., Nat. Rev. Drug Discov. 2010, 9, 203-214).

A number of approaches for improving solubility have been pursued, such as particle shape and size reduction (Yu, L. Adv. Drug Deliv. Rev. 2001, 48, 27-42; Horter, D.; Dressman, J. B. Adv. Drug Deliv. Rev. 2001, 46, 75-87), amorphous solid dispersions using appropriate polymeric excipients (Horter, D. et al., supra; Champion, J. A. et al., Proc. Natl. Acad. Sci. U. S. A. 2010, 107, 17939-17944; Tibbitt, M. W. et al., J. Am. Chem. Soc. 2016, 138, 704-717), cocrystallization (Aakeroy, C. B. et al., J. Am. Chem. Soc. 2009, 131, 17048-17049), and solubilization in co-solvents (Seedher, N.; Kanojia, M. Pharm. Dev. Technol. 2009, 14, 185-192). These approaches can outperform pure drugs, although each approach has various drawbacks mainly related to the chemical stability of the drug and physical stability against crystallization.

Thus, there still is a need for approaches to improve the drug solubility in physiological conditions and preferable a general platform for amorphous drug stabilization.

### SUMMARY

Provided herein are compositions and method for improving drug solubility and delivery. In particular, provided herein are metal-organic frameworks and their use in improving drug solubility and delivery.

The compositions and methods described herein provided improved delivery methods (e.g., oral delivery compositions and methods) for drugs with poor solubility in water. Thus, the described compositions and methods allow for oral delivery of drugs that were not previously able to be orally delivered as well as improved delivery for existing orally delivered drugs. For example, in some embodiments, provided herein is a composition, comprising: an active agent encapsulated in a metal-organic framework (MOF). The present disclosure is not limited to particular MOFs. In some exemplary embodiments, the MOF comprises zinc and a ligand (e.g., a benzene dicarboxylate ligand (e.g., a 1,4-benzene dicarboxylate ligand) or fumarate). In some embodiments, the MOF is MOF-5. In some embodiments, the agent is a poorly water soluble pharmaceutical agent (e.g., a BCS class II or IV agent). In some embodiments, the agent is curcumin, triamterene, or sulindac. In some embodiments, the agent is incorporated into the pores of the MOF after synthesis of the metal-organic framework. In some embodiments, the MOF chemically stabilizes the agent. In some embodiments, the agent is non-covalently attached to the MOF. In some embodiments, the composition is a pharmaceutical composition (e.g., tablet, capsule, or liquid). In some embodiments, the solubility of the agent in vivo or in vitro (e.g., under physiological conditions) is increased relative to the solubility of the agent in the absence of the MOF.

The present disclosure is not limited to a particular method of generating the compositions described herein. For example, in some embodiments, the composition is generated by a method, comprising: soaking the MOF in an excess of the agent in an organic solvent. In some embodiments, the soaking is performed for 1 to 10 days in the presence of shaking. In some embodiments, the composition is generated by a method, comprising: organic solvent assisted grinding of the metal-organic framework and the agent using mechanochemical grinding. In some embodiments, the composition is generated by a method, comprising: melt-loading of the agent into a metal-organic framework.

Further embodiments provide a method of delivering an active agent to a subject, comprising: administering a composition comprising the active agent encapsulated in a MOF to the subject.

Additional embodiments provide the use of a composition described herein to deliver an active agent to a subject and the use of a composition described herein to treat or prevent a disease or condition.

Also provided is a composition described herein for use in delivering an active agent to a subject.

Additional embodiments are described herein.

### DESCRIPTION OF THE FIGURES

The patent or application file contains at least one drawing executed in color. Copies of this patent or patent application publication with color drawings will be provided by the Office upon request and payment of the necessary fee.

FIG. 1 shows a pictorial representation of a drug encapsulation into MOF followed by “immediate release” of drug from drug@MOF composite via decomposition of MOF in physiological media.

FIG. 2 shows an illustration of the encapsulation of CUR (left), SUL (right), and TAT (bottom) into MOF-5 resulting in CUR@MOF-5, SUL@MOF-5, and TAT@MOF-5 composites individually.

FIG. 3 shows (a) Optical images of MOF-5 and drug@MOF-5 composites crystals. (b), (c) and (d) are PXRD patterns of drug@MOF-5 composites compared with their starting materials (MOF-5 and respective drug).

FIG. 4 shows representative CUR, CUR@MOF-5 composite and CUR/MOF-5 physical mixture (PM) dissolution profiles of in simulated gastric (SG) media (a) and phosphate buffer saline (PBS) media (b). Representation SUL, SUL@MOF-5 composite and SUL/MOF-5 physical mixture dissolution profiles in SG media (c) and PBS media (d).

FIG. 5 shows representative CUR, CUR@MOF-5 and physical mixture (PM) dissolution profiles in (a) SG and (b) PBS media. (c) Representative SUL, SUL@MOF-5, and SUL/MOF-5 PM dissolution profiles in SG media. (d) Representative TAT, TAT@MOF-5 and TAT/MOF-5 PM dissolution profiles in PBS media.

### DEFINITIONS

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human or non-human mammal subject.

As used herein, the term “diagnosed,” as used herein, refers to the recognition of a disease by its signs and symptoms (e.g., resistance to conventional therapies), or genetic analysis, pathological analysis, histological analysis, and the like.

As used herein, the terms “active agent” or “active ingredient” refer to a physiologically or otherwise active agent (e.g., pharmaceutical agent). In some embodiments, the active agent is a poorly water soluble pharmaceutical agent.

As used herein, the term “water unstable or water reactive metal-organic framework” refers to a metal organic framework that is unstable (e.g., loses structural integrity) in aqueous (e.g., physiological) conditions. In some embodiments, the metalorganic frameworks releases active agents encapsulated in the framework when it loses structural integrity or disintegrates.

As used herein, the term “effective amount” refers to the amount of a compound (e.g., a compound or formulation of the present disclosure) sufficient to effect beneficial or desired results. An effective amount can be administered in one or more administrations, applications or dosages and is not limited to a particular formulation or administration route.

As used herein, the term “co-administration” refers to the administration of at least two agent(s) (e.g., a compound or formulation of the present disclosure) or therapies to a subject. In some embodiments, the co-administration of two or more agents/therapies is concurrent. In some embodiments, a first agent/therapy is administered prior to a second agent/therapy. Those of skill in the art understand that the formulations and/or routes of administration of the various agents/therapies used may vary. The appropriate dosage for co-administration can be readily determined by one skilled in the art. In some embodiments, when agents/therapies are co-administered, the respective agents/therapies are administered at lower dosages than appropriate for their administration alone. Thus, co-administration is especially desirable in embodiments where the co-administration of the agents/therapies lowers the requisite dosage of a known potentially harmful (e.g., toxic) agent(s).

As used herein, the term “pharmaceutical composition” refers to the combination of an active agent with a carrier, inert or active, making the composition especially suitable for diagnostic or therapeutic use in vivo, in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants. (See e.g., Martin, Remington's Pharmaceutical Sciences, 15th Ed., Mack Publ. Co., Easton, PA, (1975)).

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water and industrial samples. Such examples are not however to be construed as limiting the sample types applicable to the present disclosure.

As used herein, the terms “purified” or “to purify” refer, to the removal of undesired components from a sample. As used herein, the term “substantially purified” refers to molecules that are at least 60% free, at least 65% free, at least 70% free, at least 75% free, at least 80% free, at least 85% free, at least 90% free, at least 95% free, at least 96% free, at least 97% free, at least 98% free, at least 99% free, or 100% free from other components with which they usually associated.

As used herein, the phrase “in need thereof” means that the subject has been identified as having a need for the particular method or treatment. In some embodiments, the identification can be by any means of diagnosis. In any of the methods and treatments described herein, the subject can be in need thereof. In some embodiments, the subject is in an environment or will be traveling to an environment in which a particular disease, disorder, condition, or injury is prevalent.

### DETAILED DESCRIPTION

Provided herein are compositions and method for improving drug solubility and delivery. In particular, provided herein are metal-organic frameworks and their use in improving drug solubility and delivery.

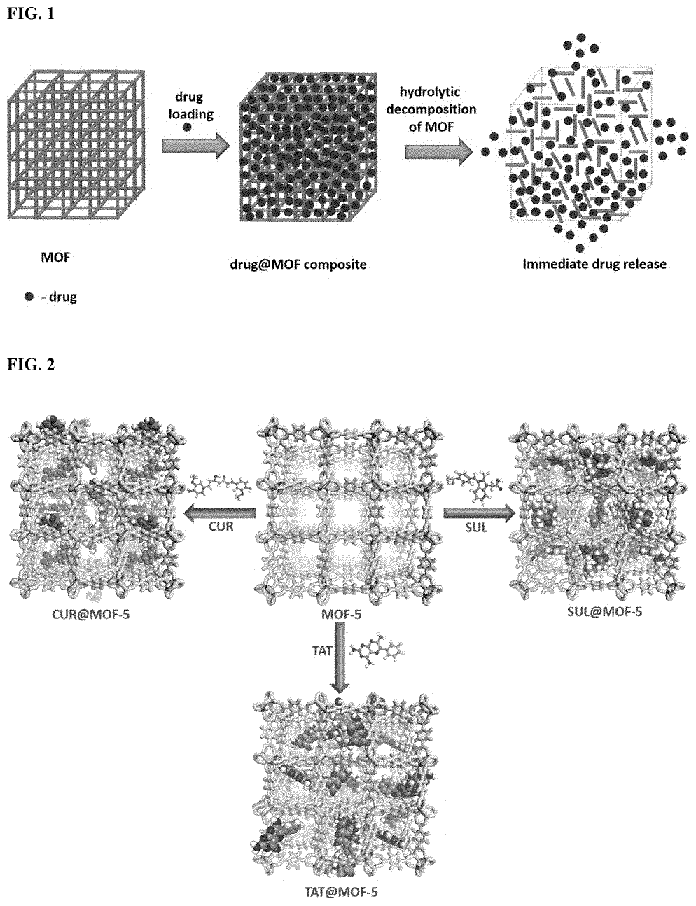

Provided herein is a general approach to improve drug solubility in physiological media by incorporating drugs into a metal-organic framework (MOF) in which the MOF acts as a “drug carrier” and undergoes rapid decomposition leading to “immediate release” of drug in the media. MOFs are composed of two major components: a metal ion or cluster of metal ions and an organic linker. The structures frequently include pores as shown in FIG. 1. FIG. 1 represents drug encapsulation followed by “immediate release” of a drug. These released drug molecules comprehensibly exhibit an increase in the free energy, which generates high supersaturation of the poorly soluble drugs.

Existing delivery methods include mesoporous silica,18 micelle and biodegradable core-shell emulsion technologies (Windbergs, M. et al., J. Am. Chem. Soc. 2013, 135, 7933-7937). In contrast, the compositions described herein utilize the “immediate release” of a drug in physiological media via MOF irreversible structural collapse in a drug@MOF composite. Depredation is leveraged to enhancing drug solubility, which provides a general approach and platform for poorly soluble drugs in pharmaceutics.

When considering an appropriate MOF to serve as an amorphous stabilizer and fast release host for a pharmaceutical, preferred properties are 1) acceptable toxicity profile, 2) reactive decomposition in appropriate media, and 3) suitable pore size to host drug targets. In exemplary compositions described herein, a zinc-based MOF-5 crystalline porous material was selected. MOF-5 is built with Zn metal cation and organic linker BDC (benzene dicarboxylate; the deprotonated form of terephthalic acid) anion and in the crystal structure (Li, H. et al., Nature 1999, 402, 276-279; herein incorporated by reference in its entirety). Zn metal is a non-toxic and biocompatible additive, supplied in dietary vitamin and mineral supplements in which Zn exists in any of the three forms such as zinc oxide, zinc acetate and zinc gluconate respectively. In addition, the organic linker terephthalic acid exhibits low toxicity and very high safe oral dose (lethal dose, LD₅₀ over 1 g/ kg) recognized in a mouse model (J. Sheehan, R.; Terephthalic Acid, Dimethyl Terephthalate, and Isophthalic Acid. In Ullmann's Encyclopedia of Industrial Chemistry). MOF-5 lacks hydrolytic stability due to cleavage of coordination bonds in humid environments (Guo, P. et al., J. Am. Chem. Soc. 2015, 137, 2651-2657). Considerably, the overall MOF-5 structure contains large pore widths approximately 12.7 Å and large Brunauer— Emmett—Teller (BET) surface area with high thermal stability (Li, H. et al., Nature 1999, 402, 276-279). Thus, MOF-5 is suitable for use as a drug carrier for improving the drug dissolution via generating supersaturation in a short time.

During experiments described herein, poorly water-soluble drugs or active agents curcumin (CUR) and sulindac (SUL) were selected as the guest molecules (FIG. 2). CUR is a hydrophobic polyphenol with dimensions ˜17.3×6.9 Å. CUR displays potential therapeutic benefits such as antioxidant, anti-tumor, anti-inflammatory, anti-malarial, anti-bacterial, anti-viral, anti-hyperglycemic activities and acts against Alzheimer's disease (Esatbeyoglu, T. et al., Chem. Int. Ed. 2012, 51, 5308-5332; Suresh, K.; Nangia, A. CrystEngComm 2018, 20, 3277-3296). CUR suffers from poor aqueous solubility, which impairs oral bioavailability. SUL is a poorly soluble non-steroidal anti-inflammatory drug with dimensions ˜13.8×7.6 Å (Shazly, G. A. Biomed. Res. Int. 2016, 3182358, 1-9).

Accordingly, provided herein are compositions comprising metal-organic frameworks comprising active agents. While the present disclosure is illustrated with MOF-5 frameworks, the present disclosure is not limited to MOF-5. Any MOF framework that increases solubility of active agents in physiological media finds use in the compositions and methods described herein. Methods of preparing MOF-5 and related structures are described, for example, in C. W. Jones, K. Tsuji and M. E. Davis, Nature 393, 52 (1998) and U.S. Pat. No. 7,196,210; each of which is herein incorporated by reference in its entirety.

In some embodiments, the MOF is a Zn/fumaric acid MOF (See e.g., Xue, M. et al., Inorg. Chem., 2009, 48 (11), 4649-4651; herein incorporated by reference in its entirety).

In some embodiments, candidate MOFs are screened for suitability for use in delivery of low solubility drugs. For example, in some embodiments, candidate MOFs are screened for their instability in aqueous (e.g., physiological) conditions using X-ray diffraction. In some embodiments, preferred MOFs exhibit a powder X-ray diffraction pattern indicative of a breakdown in the structure of the MOF after a period of time (e.g., 1 min, 3 min, 5 min, 10 min or another time) in aqueous media.

Suitable protocols for such experiments are described, for example, in Cychosz, K. A.; Matzger, A. J. Langmuir, 2010, 26(22), 17198-17202; herein incorporated in its entirety. For example, in some embodiments, MOF crystals are soaked in different water-dimethylformamide (v/v) ratios media and phase stability is monitored through powder X-ray diffraction technique. In Cychosz et al., it was first observed that the slowly intensity of the MOF diffraction peaks was decreased and then changes in powder X-ray diffraction pattern was noticed.

Similarly, phase stability of MOFs in aqueous media (including gastric media or intestinal fluid) is determined after few minutes based on changes in the intensity of the MOFs diffraction peaks to determine if a MOF is suitable for the present disclosure.

In some embodiments, the agent is incorporated into the pores of the MOF after, before, or during synthesis of the metal-organic framework. In some embodiments, the MOF is supersatured with the agent. In some embodiments, the agent is non-covalently attached to the MOF.

The present disclosure is not limited to a particular method of generating the compositions described herein. For example, in some embodiments, agents are introduced into MOFs by soaking the MOF in an excess of the agent in an organic solvent as described in Example 1. In some embodiments, the soaking is performed for 1 to 10 days in the presence of shaking.

In some embodiments, the agents are introduced into MOFs by organic solvent assisted grinding of the metal-organic framework and the agent using mechanochemical grinding (See e.g., Jean-Louis Do, J-L.; and Frisc̆ic, T. ACS Cent. Sci., 2017, 3, 13-19; herein incorporated by reference in its entirety).

In some embodiments, agents are conjugated to MOFs (See e.g., Mocniak et al., RSC Adv., 2015, 5, 83648-83656; herein incorporated by reference in its entirety).

In some embodiments, the agents are incorporate into MOFs by the melt loading method (See e.g., S. Seth, et al., Dalton Trans., 2019,48, 13483-13490; herein incorporated by reference in its entirety).

In some embodiments, the drug@MOF composites are provided as pharmaceutical compositions for oral or other routes of administration. In some embodiments, compositions for oral administration are tablets, capsules, or liquids.

The compounds described herein, optionally together with a conventional adjuvant, carrier, or diluent, may thus be placed into the form of pharmaceutical formulations and unit dosages thereof and in such form may be employed as solids, such as tablets or filled capsules, or liquids such as solutions, suspensions, emulsions, elixirs, gels or capsules filled with the same, all for oral use. Such pharmaceutical compositions and unit dosage forms thereof may comprise conventional ingredients in conventional proportions, with or without additional active compounds or principles and such unit dosage forms may contain any suitable effective amount of the active ingredient commensurate with the intended daily dosage range to be employed.

For oral administration, the pharmaceutical composition may be in the form of, for example, a tablet, capsule, suspension or liquid. The pharmaceutical composition is preferably made in the form of a dosage unit containing a particular amount of the active ingredient. Examples of such dosage units are capsules, tablets, powders, granules or a suspension, with conventional additives such as lactose, mannitol, corn starch or potato starch; with binders such as crystalline cellulose, cellulose derivatives, acacia, corn starch or gelatins; with disintegrators such as corn starch, potato starch or sodium carboxymethyl-cellulose; and with lubricants such as talc or magnesium stearate. The active ingredient may also be administered by injection as a composition wherein, for example, saline, dextrose or water may be used as a suitable pharmaceutically acceptable carrier.

The dose when using the compounds and formulations described herein can vary within wide limits and as is customary and is known to the physician, it is to be tailored to the individual conditions in each individual case. It depends, for example, on the nature and severity of the illness to be treated, on the condition of the patient, on the compound employed or on whether an acute or chronic disease state is treated or prophylaxis is conducted or on whether further active compounds are administered in addition to the compounds. Representative doses include, but not limited to, about 0.001 mg to about 5000 mg, about 0.001 mg to about 2500 mg, about 0.001 mg to about 1000 mg, 0.001 mg to about 500 mg, 0.001 mg to about 250 mg, about 0.001 mg to 100 mg, about 0.001 mg to about 50 mg and about 0.001 mg to about 25 mg. Multiple doses may be administered during the day, especially when relatively large amounts are deemed to be needed, for example 2, 3 or 4 doses. Depending on the individual and as deemed appropriate from the patient's physician or caregiver it may be necessary to deviate upward or downward from the doses described herein.

The amount of active ingredient, or an active salt or derivative thereof, for use in treatment will vary not only with the particular salt selected but also with the route of administration, the nature of the condition being treated and the age and condition of the patient and will ultimately be at the discretion of the attendant physician or clinician. In general, one skilled in the art understands how to extrapolate in vivo data obtained in a model system, typically an animal model, to another, such as a human. In some circumstances, these extrapolations may merely be based on the weight of the animal model in comparison to another, such as a mammal, preferably a human, however, more often, these extrapolations are not simply based on weights, but rather incorporate a variety of factors. Representative factors include the type, age, weight, sex, diet and medical condition of the patient, the severity of the disease, the route of administration, pharmacological considerations such as the activity, efficacy, pharmacokinetic and toxicology profiles of the particular compound employed, whether a drug delivery system is utilized, on whether an acute or chronic disease state is being treated or prophylaxis is conducted or on whether further active compounds are administered in addition to the compounds described herein and as part of a drug combination. The dosage regimen for treating a disease condition with the compounds and/or compositions is selected in accordance with a variety factors as cited above. Thus, the actual dosage regimen employed may vary widely and therefore may deviate from a preferred dosage regimen and one skilled in the art will recognize that dosage and dosage regimen outside these typical ranges can be tested and, where appropriate, may be used in the methods described herein.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations. The daily dose can be divided, especially when relatively large amounts are administered as deemed appropriate, into several, for example 2, 3 or 4 part administrations. If appropriate, depending on individual behavior, it may be necessary to deviate upward or downward from the daily dose indicated.

Non-aqueous oral suspensions/emulsions are also suitable vehicles for administration of the compositions described herein.

The compositions descried herein find use in the delivery of a variety of active agents (e.g., drugs). While the present disclosure is illustrated with curcumin (CUR) and sulindac (SUL), the present disclosure is not limited to CUR and SUL. The compositions described herein find use in the delivery of any active agent. In some embodiments, the agents are drugs that are poorly soluble under physiological conditions. In some embodiments, the agent is a BCS class II or IV agent (Mehta M (2016). Biopharmaceutics Classification System (BCS): Development, Implementation, and Growth. Wiley; herein incorporated by reference in its entirety). In some embodiments, the drug has an appropriate size that allows for loading into the pores of the selected MOF (e.g., to a high level of saturation).

Examples of BCS class II (low solubility and high permeability) drugs include, but are not limited to, carbamazepine, triamterene, efavirenz, fenofibrate, glibenclamide, metaxalone, fluconazole, griseofulvin, itraconazole, ketoconazole, azithromycin, cefdinir, cefuroximeaxetil, chloroquine, nevirapine, celecoxib, naproxen, olanzapine, oxcarbazepine, ibuprofen, ketoprofen, carvedilol, atorvastatin, cinnarizine, valsartan, ziprasidone, baicalein, montelukast, ezetimibe, sulfasalazine, clozapine, diazepam, diclofenac, flurbiprofen, haloperidol, lamotrigine, triamterene, and danazol.

Examples of BCS class IV (low solubility and low permeability) drugs include, but are not limited to, furosemide, indinavir, acyclovir, saquinavir, nelfinavir, ritonavir, lopinavir, aripiprazole, acetazolamide, cefpodoxime proxetil, azathioprine, amphotericin B, hydrochlorothiazide, colistin, chlorthalidone, chlorothiazide, cyclosporine A, paclitaxel, docetaxel, famotidine, naftopidil, etravirine and entacapone.

In some embodiments, the compositions described herein find use in research, screening, and therapeutic application. For example, in some embodiments, formulations find use in the treatment, preventions, or management of any number of diseases, symptoms, and conditions.

### EXPERIMENTAL

### Example 1

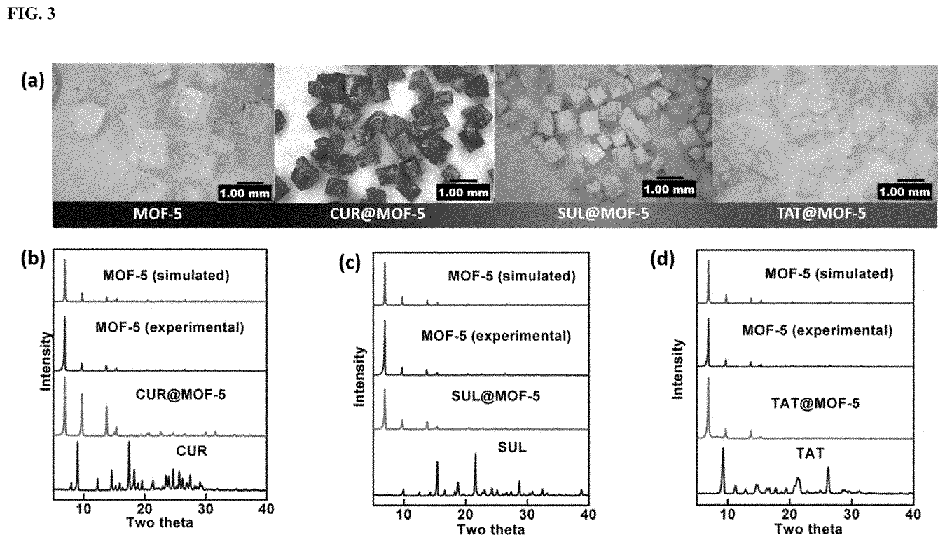

Drug molecules were encapsulated via post-synthetic incorporation by soaking activated MOF-5 crystals in excess of a drug in methylene chloride (for CUR) and acetonitrile (for SUL) for 10 days on a shaker. The drug@MOF-5 composite crystals were removed by filtration and washed twice with the same solvent used for the incorporation to eliminate residual drug molecules on the outside surface of MOF-5. The crystals were activated and analyzed by powder X-ray diffraction (PXRD) and spectroscopy (NMR, infrared and Raman) techniques. Optical microscopy (FIG. 3) showed the dramatic color change upon encapsulation of drug. CUR@MOF-5 composite becomes a brick red and SUL@MOF-5 composite becomes yellow. PXRD patterns of drug@MOF-5 (CUR/SUL) composites demonstrate that the MOF-5 phase stability and crystallinity is retained after encapsulation of drug molecules (FIG. 3c and d). No diffraction peaks associated with drug molecules were observed. Consistent with the pores being too small to support aggregation into a crystalline form. Proton NMR spectra of drug@MOF-5 composites, after digestion in acidic DMSO-d₆ revealed that drugs do not degrade upon laoding. The vibrational spectra (IR and Raman) of drug@MOF-5 composites show similarities and small peak shift differences in MOF-5 characteristic peaks as well as a drug when compared to the starting components. These spectral techniques support the phase stability of MOF-5 and drug molecules incorporated into the pores of MOF-5. Specifically, in SUL@MOF-5 composite, the carboxylic acid functional group C=0 (carbonyl) largely red shifted (1676 cm^{'1}) in IR spectra compared to the pure SUL (1697 cm⁻¹). Also in Raman spectra, the same C=0 peak of SUL carboxylic acid and carboxylate of MOF-5 buried and red-shifted compared to the parent compounds. This C=0 peak shift differences in both IR and Raman spectra is consistent with significant attractive drug-MOF interactions in the composite . . .

Weight percentage (wt%) of the incorporated drug in a drug@MOF_5 composite was determined using UV-vis spectroscopy. The CUR wt % in CUR@MOF-5 and SUL wt % in SUL@MOF-5 composite measured at λmax 420 nm (for CUR) and 330 nm (for SUL) respectively. Drug encapsulation was found to be 7.7% for CUR and 22.4% for SUL. Usually, the loading capacity into MOF is governed by various factors including pore size, shape and host affinity. Furthermore, the activated drug@MOF-5 composites sorption analysis revealed that the BET surface area decreases in both CUR@MOF-5 composite (2777 m²g⁻¹) and SUL@MOF-5 composite (1969 m²g⁻¹) than pure MOF-5 (3425 m²g⁻¹), which shows that pores of MOF-5 are occupied by drug molecules.

Prior to drug release and dissolution studies, MOF-5 decomposition was confirmed by soaking excess MOF-5 crystals in both simulated gastric (SG) and phosphate buffer (PBS) media at 37° C. The undissolved material from these media PXRD analysis validates that neutral terephthalic acid precipitates out in SG media whereas a Zn-terephthalate-dihydrate salt precipitate including other decomposition products such as hydrated metal salts or presence different of metal ion state are obtained in PBS media. Similarly, the drug@MOF-5 composites decomposition behavior was akin to pure MOF-5. Notably, SUL@MOF-5 composite decomposition in SG media resulted in SUL crystallized as form I (monoclinic) polymorph not as starting material (form II, orthorhombic) confirmed by PXRD analysis (Grzesiak, A. L.; Matzger, A. J. J. Pharm. Sci. 2007, 96, 2978-2986).

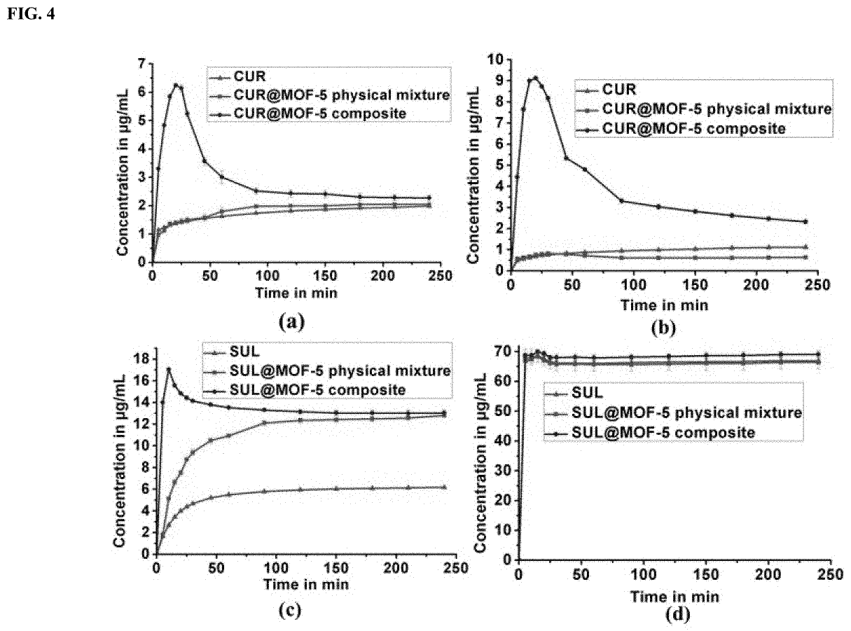

To determine the dissolution and supersaturation behavior in SG and PBS media of all CUR, SUL, CUR@MOF-5 and SUL@MOF-5 composites and their physical mixture samples, UV-vis spectroscopy (which is applied to monitor the dissolution process) was used. Concentration of CUR and SUL at 37° C. was determined. FIGS. 4a-d illustrate the solubility of pure CUR, CUR@MOF-5 composite and physical mixture and pure SUL, SUL@MOF-5 composite and physical mixture in SG and PBS media respectively. The shape of the drug concentration vs. time curves for CUR@MOF-5 composite in both media indicates that CUR rapidly dissolved followed by slower crystallization (precipitation) behavior. Because the immediate release of CUR molecules (from CUR@mof-5 composite) led to rapid dissolution, which generates high supersaturation in the first 60 min. The maximum concentration (Cmax) 6.24 μg/mL in SG media and 9.13 μg/mL in PBS media were observed and these values demonstrated that CUR@MOF-5 composite achieved much higher concentration than pure CUR solubility. Subsequently, a crystallization process occurs accompanied by a decrease in CUR concentration in both media.

It is anticipated that supersaturation is thermodynamically unstable state due to the high free energy of the system and defined as an increase in the free drug concentration in media above its saturation solubility. Unexpectedly, Cₘₐₓ and AUC values of CUR@MOF-5 composite (Table 1) are much greater than pure CUR. Similarly, CUR/MOF-5 physical mixture dissolution profile follows the pure CUR in both media, indicating that encapsulation is critical for performance.

Sulindac is a poorly water-soluble weak carboxylic acid which displays pH-dependent solubility and increases with increase with pH. Thus, the dissolution behavior of SUL@MOF-5 relative to crystalline SUL was dependent on dissolution media pH. During dissolution of SUL@MOF-5 composite and physical mixture in SG media, pH slightly changed because of MOF-5 decomposed products. This change in pH lead to substantial changes in SUL solubility and supersaturation. The SUL@MOF-5 composite burst releases SUL molecules, which generate the high supersaturation in the first 10 min of dissolution. The concentration was 17.05 μg/mL. As the dissolution continues, the concentration of SUL declines due to SUL crystallization, which equilibrated at 13.04 μg/mL and matches MOF-5/SUL physical mixture solubility. However, there is no effect of immediate release of SUL from SUL@MOF-5 composite in PBS media because of pure SUL higher solubility. Therefore, all the tested solids SUL, SUL@MOF-5 composite and SUL/MOF-5 physical mixture, are fully dissolved in this media and exhibit similar dissolution behavior (Table 1).

In summary, drugs exhibited a high supersaturation behavior in drug@MOF composite system, which is beneficial to improve the solubility of pharmaceuticals. The results demonstrate that poorly water-soluble pharmaceuticals (CUR/SUL) dramatically improved their solubility via the combination of a quickly dissolving high supersaturation with a slower crystallization in physiological media which is potential for onset therapeutic activity and effective to the extent oral absorption of a drug.

TABLE 1
Cₘₐₓ and AUC₀₋₄ values (Average (Standard Error of the
Mean)) of CUR, SUL, CUR@MOF-5 and SUL@MOF-5, composites
and their physical mixtures (PM) in SG and/or PBS media.

| | Cₘₐₓ (μg/mL) | | AUC₀₋₄ (mg*h/mL) | |
|---|---|---|---|---|
| compound | SG media | PBS media | SG media | PBS media |
| CUR | 1.99(4) | 1.12(7) | 0.41(6) | 0.22(3) |
| CUR/MOF-5 PM | 2.04(4) | 0.81(5) | 0.44(5) | 0.15(3) |
| CUR@MOF-5 | 6.24(3) | 9.13(3) | 0.70(6) | 0.93(9) |
| SUL | 6.01(6) | 68.65(91) | 1.32(2) | 15.82(21) |
| SUL/MOF-5 PM | 12.80(14) | 69.96(62) | 2.70(4) | 16.28(24) |
| SUL@MOF-5 | 17.10(5) | 68.34(1.7) | 3.20(41) | 15.70(21) |

### Example 2

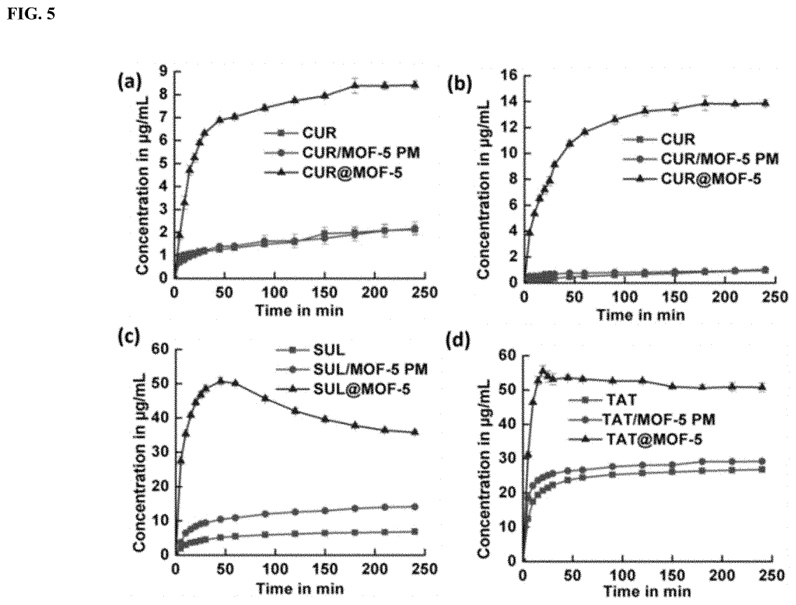

This Example described further characterization of a variety of drugs in MOF-5. Methyl cellulose polymer was included in both SG and PBS) dissolution media to inhibit precipitation of drug molecules at high supersaturation and to serve as a proxy for what would be an extensive formulation process of this supersaturating drug delivery system. The shape of the drug concentration vs. time curves for CUR@MOF-5 in both media indicates that CUR undergoes rapid dissolution and maintains high supersaturation. Immediate release of CUR molecules from CUR@MOF-5 via MOF hydrolytic decomposition leads to rapid dissolution which generates high supersaturation in the first 60 min (FIGS. 5a and b). A maximum concentration (Cmax) of 8.5 μg/mL in SG media and 13.9 μg/mL in PBS media are observed and demonstrating that CUR@MOF-5 achieves a much higher CUR concentration upon dissolution than pure CUR (Table 2). This immediate drug release from drug@MOF-5 composite is in contrast with controlled drug release from an amorphous drug@MOF composite with a water stable framework (C. Orellana-Tavra, et al., Chem. Commun. 2015, 51, 13878). A CUR/MOF-5 PM displays a dissolution profile similar to pure CUR in both media, indicating that encapsulation is critical for performance. Moreover, CUR@MOF-5 achieves supersaturation significantly greater than that generated during the dissolution of CUR polymeric amorphous solid dispersions (S. Onoue et al., J. Pharm. Sci. 2010, 99, 1871).

Similarly, immediate release of SUL from SUL@MOF-5 in SG media generates high supersaturation in the first 60 min of dissolution with a maximum concentration of 50.7 μg/mL. As dissolution continues, the concentration of SUL declines slowly due to SUL precipitation from media. Nonetheless, Cₘₐₓ and AUC values of SUL@MOF-5 are much greater than pure SUL (FIG. 5c). Furthermore, SUL@MOF-5 achieves superior supersaturation as compared to SUL polymeric amorphous solid dispersions, which exhibit a Cₘₐₓ of <33 μg/mL in SG (J. Maclean et al., J. Pharm. Sci. 2011, 100, 3332).

Furthermore, another example, triamterene (TAT) drug was tested by applying this drug delivery system. TAT is a potassium-sparing diuretic drug with dimensions ˜11.8×7.6 A that exhibits poor solubility in neutral solutions and intestinal pH (W. J. Ma, et al., Mol. Pharm. 2013, 10, 4698).TAT drug was encapsulated via post-synthetic incorporation by soaking activated MOF-5 crystals in excess of a drug in acetonitrile solvent and encapsulation was found to be 34.0 wt % in a TAT@MOF-5. No diffraction peaks associated with TAT is observed, consistent with the pores being too small to support aggregation into a crystalline form (FIG. 3d). Immediate release of TAT from TAT@MOF-5 composite in PBS media generates high supersaturation in the first 20 min of dissolution with a maximum concentration of up to 55.5 μg/mL which demonstrates that TAT@MOF-5 achieves enhanced concentration upon dissolution than pure TAT (FIG. 5d). A TAT/MOF-5 PM displays a dissolution profile similar to pure TAT.

TABLE 2
Representative drugs, drug@MOF-5 composites
and their PM Cₘₐₓ and AUC₀₋₄ values (Average (Standard
Error of the Mean)) in SG and/or PBS media.

| | Cₘₐₓ (μg/mL) | | AUC₀₋₄ (mg*h/mL) | |
|---|---|---|---|---|
| compound | SG media | PBS media | SG media | PBS media |
| CUR | 2.12(21) | 0.95(4) | 0.39(6) | 0.15(2) |
| CUR/MOF-5 PM | 2.16(3) | 1.03(1) | 0.39(6) | 0.19(3) |
| CUR@MOF-5 | 8.45(19) | 13.87 (27) | 1.75(2) | 2.88(4) |

| | SG media | SG media |
|---|---|---|
| SUL | 6.84(7) | 1.38(2) |
| SUL/MOF-5 PM | 14.06(6) | 2.83(5) |
| SUL@MOF-5 | 50.73(41) | 9.92(13) |
| | PBS media | PBS media |
| TAT | 26.89(8) | 5.89(9) |
| TAT/MOF-5 PM | 29.67(6) | 8.74(9) |
| TAT@MOF-5 | 55.50(2) | 15.25(17) |

All publications and patents mentioned in the present application are herein incorporated by reference. Various modification and variation of the described methods and compositions of the disclosure will be apparent to those skilled in the art without departing from the scope and spirit of the disclosure. Although the disclosure has been described in connection with specific preferred embodiments, it should be understood that the disclosure as claimed should not be unduly limited to such specific embodiments. Indeed, various modifications of the described modes for carrying out the disclosure that are obvious to those skilled in the relevant fields are intended to be within the scope of the following claims.

1. A method of delivering triamterene to a subject, comprising:
administering a composition comprising triamterene encapsulated in a water unstable or water reactive metal-organic framework to said subject under conditions such that said metal-organic framework undergoes rapid decomposition and said triamterene is immediately released from said metal-organic framework.

2. The method of claim 1, wherein said administering treats a disease or condition.

3. The method of claim 1, wherein the aqueous solubility of said drug is increased relative of the aqueous solubility of said drug when not encapsulated in said metal-organic framework.

4. The method of claim 1, wherein said metal-organic framework comprises a dietary metal.

5. The method of claim 1, wherein said metal-organic framework has a pore size suitable to hold said triamterene.

6. The method of claim 1, wherein said metal-organic framework comprises zinc and a ligand.

7. The method of claim 6, wherein said ligand is benzene dicarboxylate or fumarate.

8. The method of claim 7, wherein said benzene dicarboxylate ligand is a 1,4-benzene dicarboxylate ligand.

9. The method of claim 8, wherein metal-organic framework is metal-organic framework 5.

10. The method of claim 1, wherein said agent is incorporated into the pores of said metal-organic framework after synthesis of said metal-organic framework.

11. The method of claim 1, wherein said metal organic framework generates a supersaturated solution of said triamterene.

12. The method of claim 1, wherein said triamterene is non-covalently attached to said metal-organic framework.

13. The method of claim 1, wherein said composition is a pharmaceutical composition.

14. The method of claim 13, wherein said pharmaceutical composition is a tablet, capsule, or suspension.
